(19) United States
(12) Patent Application Publication
(10) Pub. No.: US 2013/0123357 A1
(43) Pub. Date: May 16, 2013

(54) Methods for Providing Enhanced Resveratrol Activity Using 4-Acetoxy-Resveratrol

(71) Applicants: Edwin Douglas Lephart, Orem, UT (US); Merritt B. Andrus, Lindon, UT (US)

(72) Inventors: Edwin Douglas Lephart, Orem, UT (US); Merritt B. Andrus, Lindon, UT (US)

(73) Assignee: BRIGHAM YOUNG UNIVERSITY, Provo, UT (US)

(21) Appl. No.: 13/641,449

(22) PCT Filed: Apr. 13, 2011
(86) PCT No.: PCT/US11/32306

§ 371 Date: Jan. 25, 2013

Related U.S. Application Data
(60) Provisional application No. 61/323,608, filed on Apr. 13, 2010.

(51) Int. Cl. A61K 31/216

(52) U.S. Cl. CPC A61K 31/216
USPC 514/546

(57) ABSTRACT

The present invention relates to a method of providing enhanced resveratrol activity comprising: administering a therapeutically effective amount of 4-acetoxy-resveratrol for the treatment of and preventing physiological and pathophysiological conditions mediated by (1) sirtuins, (2) estrogen and anti-estrogen hormone actions and (3) chemical interventions important for male and female health, aging, anti-aging and age-related disorders.

### REFERENCE TO EARLIER FILED APPLICATION

This application claims the benefit under 35 U.S.C. §119(e) of U.S. Provisional Patent Application No. 61/323,608, filed Apr. 13, 2010, and titled “METHODS FOR PROVIDING ENHANCED RESVERATROL ACTIVITY USING 4-ACETOXY-RESVERATROL,” which is incorporated, in its entirety, by this reference.

### FEDERALLY SPONSORED RESEARCH OR DEVELOPMENT

This invention was made with Government support under Grant No. GM057275, awarded by the National Institute of Health (NIGMS) and Grant No. NR12004-01811, awarded by the U.S. Dept. of Agriculture (USDA).

### BACKGROUND

### Technical Field

The present invention relates to methods of treating biological conditions involving sirtuin enzymes with ester analog compounds of resveratrol that activate sirtuin enzymes, and more particularly to medicinal and cosmetic uses of 4-acetoxy-resveratrol.

The sirtuins are a class of NAD⁺-dependent protein deacetylase enzymes that regulate a wide variety of cellular activities that promote cell survival and extend lifespan in response to environmental stress. Sirtuins exert their effect by removing acetyl groups from certain target proteins, including histones, transcription factors and cytosolic acetyl CoA synthetase, in the presence of oxidized nicotinamide adenine dinucleotide (NAD⁺). For example, the yeast sirtuin enzyme Sir2 (silent information regulator 2), originally identified for its role in silencing transcription of DNA, has also been shown to promote cell survival in response to caloric restriction. Similarly, in C. elegans, the sirtuin enzyme SIR-2.1 has been shown to extend lifespan. In mammalian cells, the sirtuin enzyme SIRT1 (a homolog of the yeast Sir2 and C. elegans SIR-2.1 enzymes) deacetylates the tumor suppressor p53 to promote cell survival. Sirtuins therefore appear to be activated as part of a beneficial cellular response to stress, resulting in cell survival and extended lifespan. Activators of sirtuins may therefore be beneficial in effecting fundamental cellular processes that protect cells from stress and prevent or treat age-related diseases, and lengthen healthy life.

Resveratrol (3,5,4′-trihydroxy-trans-stilbene) is a polyphenol compound, known to be the most potent activator of sirtuins. As a stilbene phytoalexin, resveratrol continues to receive increasing attention for its role in mitigation of numerous and diverse human pathological processes including inflammation, atherosclerosis, and carcinogenesis. Resveratrol is known for its activity as an antioxidant, cyclooxygenase inhibitor, lipid modifier, platelet aggregation inhibitor and vasodilator, inhibitor of tumor initiation, promotion and progression, neuroprotector, and antiviral compound.

Resveratrol is an especially abundant component of red wines produced from grapes grown in cooler climates where plants are under stress from heavy disease pressure. Indeed, the consumption of red wine containing resveratrol is believed to be responsible for the surprisingly normal lifespan of the French, despite their heavy consumption of fatty foods that cause heart disease, a phenomenon referred to as the “French Paradox.” Resveratrol, however, is not an optimal sirtuin activator. Its practical use is also limited due to difficult isolation and stereo-selectively of extracts obtained from plant sources. More significantly, resveratrol is highly unstable due to its potential for oxidation, resulting in the formation of unstable radicals and quinones, and requiring its isolation be carried out from impure mixtures containing multiple components.

### BRIEF SUMMARY

The present invention provides a resveratrol analog compound, 4-acetoxy-resveratrol (also sometimes referred to as 4′-acetoxy-resveratrol or 4AR), which shows significantly enhanced resveratrol activity.

4AR has the following chemical formula:

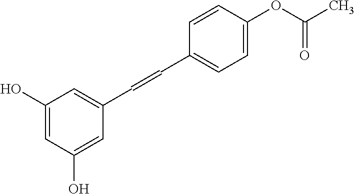 [SMILES: CC(=O)Oc1ccc(C=Cc2cc(O)cc(O)c2)cc1]

In some embodiments, methods for treating and preventing physiological and pathophysiological conditions mediated by (1) sirtuins, (2) estrogen and anti-estrogen hormone actions, and (3) chemical interventions important for male and female health, aging, anti-aging and age-related disorders are disclosed. This includes conditions and disorders such as obesity, weight control, diabetes, insulin-resistance, cardiovascular disease, atherosclerosis, bone loss or osteoporosis, Alzheimer's disease and neurodegeneration, skin disorders and skin cancer, prostate cancer and other forms of carcinogenesis and benign prostatic hyperplasia or BPH.

In some embodiments, the invention provides a method of enhancing resveratrol activity comprising administering a therapeutically effective amount of 4-acetoxy-resveratrol.

In some embodiments, the invention provides a method of treating or preventing conditions mediated by sirtuins, comprising administering a therapeutically effective amount of 4-acetoxy-resveratrol.

In some embodiments, the invention provides a method of treating or preventing conditions mediated by estrogen and anti-estrogen hormone actions comprising administering a therapeutically effective amount of 4-acetoxy-resveratrol.

In some embodiments, the invention provides a method of treating or preventing a skin condition comprising administering a therapeutically effective amount of 4-acetoxy-resveratrol.

In some embodiments, the invention provides a method wherein 4-acetoxy-resveratrol is administered in a topical cream, lotion, gel or other cosmetic formulation, optionally including another resveratrol ester analog or combination of resveratrol ester analogs in concentrations between 0.3% and 12%.

In some embodiments, the invention provides a method wherein 4-acetoxy-resveratrol is administered in a dermal patch delivery system, optionally including another resveratrol ester analog or combination of resveratrol ester analogs in a concentration between 1% and 30%.

In some embodiments, the invention provides a method, wherein 4-acetoxy-resveratrol is administered in an oral dosage, optionally including another resveratrol ester analog or combination of resveratrol ester analogs in an amount between 10 mg and 1600 mg.

In some embodiments, the invention provides a method wherein 4-acetoxy-resveratrol is administered by injection, optionally including another resveratrol ester analog or combination of resveratrol ester analogs in an amount between 5 mg and 1600 mg.

In some embodiments, the invention provides a method wherein 4-acetoxy resveratrol is administered in a food product, optionally including another resveratrol ester analog or combination of resveratrol ester analogs in a concentration between 0.1% and 10%. In some embodiments, the food product is selected from an energy bar, cereal, beverage, energy drink, dip, yogurt, gum, and candy.

### FIGURE

FIG. 1 depicts histological slides of in vitro Human Dermal Tissue Model in gene array studies.

### DETAILED DESCRIPTION

Resveratrol and its analogs, including 4AR, bind to multiple substrates and, consequently, have multiple biological functions. For example, 4AR functions as an antioxidant, a cyclooxygenase inhibitor, and a lipid modifier. They positively influence fatty acid synthesis, prevent inflammation, promote longevity in cells, extend the life span (anti-aging) of organisms, promote dermal molecules or components of skin (such as increasing collagen deposition in human fibroblasts and positively enhance many other dermal components such as elastin, elastase, matrix metalloproteinases, collagenases, glycoaminoglycans, and hyaluronic acid at the epidermal/dermal junction).

Additionally, 4AR can bind the abundant distribution of beta estrogen receptors in the keratinocytes of the epidermis and fibroblasts in the dermis that also have a positive influence on skin parameters and enhanced dermal health and act as an anti-aging factor in skin.

Preparation of 4AR

4′-Acetoxy-Resveratrol (referred to herein as 4AR) can be synthesized in five steps beginning with resorcyclic acid, as described in PCT/US05/02229 (WO 2005/069998) which is hereby incorporated by reference in its entirety.

Compounds

The methods of the present invention use the compound 3,5-dihydroxy-4′-acetoxy stilbene (also referred to as 4′-acetoxy resveratrol, 4-acetoxy resveratrol, and 4AR). 4AR is capable of forming both pharmaceutical and cosmetically acceptable formulations.

Pharmaceutical Uses

The compositions disclosed herein can be prepared and administered in a wide variety of oral and parenteral dosage forms. Thus, the compositions can be administered by injection, that is, intravenously, intramuscularly, intracutaneously, subcutaneously, intraduodenally, or intraperitoneally. Also, the compositions can be administered by inhalation, for example, intranasally. Additionally, the compositions can be administered transdermally. It will be obvious to those skilled in the art that the following dosage and application forms may comprise as the active component, either 4-acetoxy resveratrol or a corresponding pharmaceutically or cosmetically acceptable salt of 4-acetoxy-resveratrol which has the following structure:

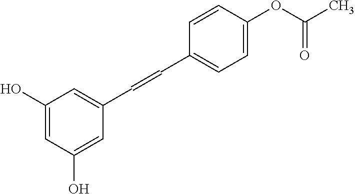 [SMILES: CC(=O)Oc1ccc(C=Cc2cc(O)cc(O)c2)cc1]

In some embodiments, 4-acetoxy-resveratrol is in the cis configuration. In some embodiments, 4-acetoxy-resveratrol is in the trans configuration. In some embodiments, 4-acetoxy-resveratrol exists in both the cis and trans configurations.

As used herein, the term “resveratrol activity” includes biological activity which 4AR and/or resveratrol exhibits. For example, resveratrol activity includes biological processes involved in inflammation, atherosclerosis, carcinogenesis, or as an antioxidant, cyclooxygenase inhibitor, lipid modifier, platelet aggregation inhibitor and vasodilator, inhibitor of tumor initiation, promotion and progression, neuroprotector, and antiviral compound. Resveratrol activity also includes enhancing skin health and other conditions pertaining to aging, anti-aging, and age-related disorders.

For preparing pharmaceutical compositions from the compounds of the present invention, pharmaceutically acceptable carriers can be either solid or liquid. Solid form preparations include powders, tablets, pills, capsules, cachets, suppositories, and dispersible granules. A solid carrier can be one or more substances which may also act as diluents, flavoring agents, binders, preservatives, tablet disintegrating agents, or an encapsulating material.

In powders, the carrier is a finely divided solid which is in a mixture with the finely divided active component.

In tablets, the active component is mixed with the carrier having the necessary binding properties in suitable proportions and compacted in the shape and size desired.

The powders and tablets preferably contain from about five or about ten to about seventy percent of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other carriers, is surrounded by a carrier, which is thus in association with it. Similarly, cachets and lozenges are included. Tablets, powders, capsules, pills, cachets, and lozenges can be used as solid dosage forms suitable for oral administration.

For preparing suppositories, a low melting wax, such as a mixture of fatty acid glycerides or cocoa butter, is first melted and the active component is dispersed homogeneously therein, as by stirring. The molten homogeneous mixture is then poured into conveniently sized molds, allowed to cool, and thereby to solidify.

Liquid form preparations include solutions, suspensions, and emulsions, for example, water or water propylene glycol solutions. For parenteral injection liquid preparations can be formulated in solution in aqueous polyethylene glycol solution.

Aqueous solutions suitable for oral use can be prepared by dissolving the active component in water and adding suitable colorants, flavors, stabilizing and thickening agents as desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, table, cachet, or lozenge itself, or it can be the appropriate number of any of these in packaged form.

The quantity of active component in a unit dose preparation may be varied or adjusted from 0.1 mg to 1000 mg preferably 0.5 mg to 100 mg according to the particular application and the potency of the active component. The composition can, if desired, also contain other compatible therapeutic agents.

Cosmetic Uses

For preparing cosmetic compositions from the processes and compounds of the present invention, cosmetically acceptable vehicles or carriers facilitate distribution when the composition is applied to the skin. The vehicle may be aqueous, anhydrous or an emulsion. Preferably, the compositions are aqueous or an emulsion, especially water-in-oil or oil-in-water emulsion. Water when present will be in amounts which may range from about 5 to about 99%, in some embodiments from about 20 to about 70%, and in some embodiments between about 35 and 60% by weight.

Besides water, relatively volatile solvents may also serve as carriers within compositions of the present invention, such as transcutol. Most preferred are monohydric C₁-C₃ alkanols. These include ethyl alcohol, methyl alcohol and isopropyl alcohol. The amount of monohydric alkanol may range from about 1 to about 70%, in some embodiments from about 10 to about 50%, and in some embodiments between about 15 and about 40% by weight.

Emollient materials may also serve as cosmetically acceptable carriers. These may be in the form of silicone oils and synthetic esters. Amounts of the emollients may range anywhere from about 0.1 to about 50%, in some embodiments between about 1 and about 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, in some embodiments from 4 to 5 silicon atoms. Linear volatile silicone materials generally have viscosities less than about 5 centistokes at 25° C., while cyclic materials typically have viscosities of less than about 10 centistokes. Nonvolatile silicone oils useful as an emollient material include polyalkyl siloxanes, polyalkylaryl siloxones, and polyether siloxane copolymers. The essentially non-volatile polyalkyl siloxanes useful herein include, for example, polydimethyl siloxanes with viscosities of from about 5 to about 25 million centistokes at 25° C. Among the preferred non-volatile emollients useful in the present compositions are the polydimethyl siloxanes having viscosities from about 10 to about 400 centistokes at 25° C.

Among the ester emollients are: (1) alkenyl or alkyl esters of fatty acids having 10 to 20 carbon atoms. Examples thereof include isoarachidyl neopentanoate, isononyl isonanonoate, oleyl myristate, oleyl stearate, and oleyl oleate; (2) ether-esters such as fatty acid esters of ethoxylated fatty alcohols; (3) polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters; (4) wax esters such as beeswax, spermaceti, myristyl myristate, stearyl stearate and arachidyl behenate; and, (5) sterols esters, of which cholesterol fatty acid esters are examples thereof; fatty acids having from 10 to 30 carbon atoms may also be included as cosmetically acceptable carriers for compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be employed as cosmetically acceptable carriers in compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylen polyols and their derivatives, including propylene glycol, dipropylene glycol, polypropylene glycol, polyethylene glycol and derivatives thereof, sorbitol, hydroxypropyl sorbitol, hexylene glycol, 1,3-dibutylene glycol, 1,2,6-trihexanetriol, ethoxylated glycerol, propoxylated glycerol and mixtures thereof. In some embodiments the humectant is propylene glycol or sodium hyaluronate. The amount of humectant may range anywhere from about 0.5 to about 30%, in some embodiments between about 1 and about 15% by weight of the composition.

Thickeners may also be utilized as part of the cosmetically acceptable carrier of compositions according to the present invention. Typical thickeners include crosslinked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, in some embodiments from 0.001 to 1%, and in some embodiments from 0.01 to 0.5% by weight.

Collectively the water, solvents, silicones, esters, fatty acids, humectants and/or thickeners will constitute the cosmetically acceptable carrier in amounts from about 1 to 99.9%, in some embodiments from 80 to 99% by weight.

An oil or oily material may be present, together with an emulsifier to provide either a water-in-oil emulsion or an oil-in-water emulsion, depending largely on the average hydrophilic-lipophilic balance (HLB) of the emulsifier employed.

Surfactants may also be present in cosmetic compositions. Total concentration of the surfactant can range from about 0.1 to about 40%, in some embodiments from 1 to 20%, and in some embodiments from 1 to 5% by weight of the composition. The surfactant may be selected from the group consisting of anionic, nonionic, cationic and amphoteric actives. In some embodiments, nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

Sunscreens may be present in cosmetic compositions of the present invention. Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation.

Many cosmetic compositions, especially those containing water, must be protected against the growth of potentially harmful microorganisms. Preservatives are, therefore, often necessary. Suitable preservatives include alkyl esters of p-hydroxybenzoic acid, hydantoin derivatives, propionate salts, and a variety of quaternary ammonium compounds. In some embodiments, a preservative can be included selected from methyl paraben, propyl paraben, phenoxyethanol and benzyl alcohol. Preservatives can be present in amounts ranging from about 0.1% to 2% by weight of the composition.

Powders may be incorporated into the cosmetic composition of the invention. These powders include chalk, talc, Fuller's earth, kaolin, starch, smectites clays, chemically modified magnesium aluminum silicate, organically modified montmorillonite clay, hydrated aluminum silicate, fumed silica, aluminum starch octenyl succinate and mixtures thereof.

The composition can be packaged in a suitable container to suit its viscosity and intended use by the consumer. For example, a lotion or fluid cream can be packaged in a bottle or a roll-ball applicator, or a capsule, or a propellant-driven aerosol device or a container fitted with a pump suitable for finger operation. When the composition is a cream, it can simply be stored in a non-deformable bottle or squeeze container, such as a tube or a lidded jar.

Cosmetic formulations can be used in various fields. For example, the following preparations especially come into consideration: skin-care preparations, e.g. skin-washing and cleansing preparations in the form of tablet-form or liquid soaps, synthetic detergents or washing pastes; bath preparations, e.g. liquid (foam baths, milks, shower preparations) or solid bath preparations, e.g. bath cubes and bath salts; skin-care preparations, e.g. skin emulsions, multi-emulsions or skin oils; cosmetic personal care preparations, e.g. facial make-up in the form of day creams or powder creams, face powder (loose or pressed), rouge or cream make-up, eye-care preparations, e.g. eyeshadow preparations, mascara, eyeliner, eye creams or eye-fix creams; lip-care preparations, e.g. lipstick, lip gloss, lip contour pencils, nail-care preparations, such as nail varnish, nail varnish remover, nail hardeners, or cuticle removers; intimate hygiene preparations, e.g. intimate washing lotions or intimate sprays; foot-care preparations, e.g. foot baths, foot powders, foot creams or foot balsams, special deodorants and antiperspirants or callous-removing preparations; light-protective preparations, such as sun milks, lotions, creams, oils, sun blocks or tropicals, pre-tanning preparations or after-sun preparations; skin-tanning preparations, e.g. self-tanning creams; depigmenting preparations, e.g. preparations for bleaching the skin or skin-lightening preparations; insect-repellents, e.g. insect-repellent oils, lotions, sprays or sticks; deodorants, such as deodorant sprays, pump-action sprays, deodorant gels, sticks or roll-ons; antiperspirants, e.g. antiperspirant sticks, creams or roll-ons; preparations for cleansing and caring for blemished skin, e.g. synthetic detergents (solid or liquid), peeling or scrub preparations or peeling masks; hair-removal preparations in chemical form (depilation), e.g. hair-removing powders, liquid hair-removing preparations, cream- or paste-form hair-removing preparations, hair-removing preparations in gel form or aerosol foams; shaving preparations, e.g. shaving soap, foam shaving creams, non-foaming shaving creams, foams, gels, preshave preparations for dry shaving, aftershaves or aftershave lotions; fragrance preparations, e.g. fragrances (eau de Cologne, eau de toilette, eau de parfum, parfum de toilette, perfume), perfume oils or perfume creams; dental-care, denture-care and mouth-care preparations, e.g. toothpastes, gel tooth-pastes, tooth powders, mouthwash concentrates, anti-plaque mouthwashes, denture cleaners or denture fixatives; and, cosmetic hair-treatment preparations, e.g. hair-washing preparations in the form of shampoos and conditioners, hair-care preparations, e.g. pre-treatment preparations, hair tonics, styling creams, styling gels, pomades, hair rinses, treatment packs, intensive hair treatments, hair-structuring preparations, e.g. hair-waving preparations for permanent waves (hot wave, mild wave, cold wave), hair-straightening preparations, liquid hair-setting preparations, hair foams, hair sprays, bleaching preparations, e.g. hydrogen peroxide solutions, lightening shampoos, bleaching creams, bleaching powders, bleaching pastes or oils, temporary, semi-permanent or permanent hair colorants, preparations containing self-oxidizing dyes, or natural hair colorants, such as henna or camomile.

The compounds and compositions disclosed herein have significantly improved biological activity and can be stored over a long period without alteration.

The following non-limiting examples illustrate methods for preparing the compounds and compositions disclosed herein.

Nutritional Uses

When the composition is incorporated into various media such as foods, it may simply be orally ingested. The food can be a dietary supplement such as a snack or wellness dietary supplement or, especially for animals, comprise the nutritional bulk of a feedstock. The nutritional product may also be selected from an energy bar, cereal, beverage, energy drink, dip, yogurt, gum, and candy.

Mode or Route of Administration

The disclosed compositions may be administered in a topical cream, lotion, gel or other cosmetic formulation, where a resveratrol ester such as 4AR may be present in the form of a single analog or combined with other resveratrol ester analogs, in concentrations of about 0.3% to about 12% such as at least 0.3% to 2% or about 1.5% to 12%.

In some embodiments, the disclosed compositions may be administered in the form of a dermal patch delivery system to maintain systemic resveratrol ester levels, as a single analog or a combination of resveratrol ester analogs, in concentrations of from about 1% to about 30%. In some embodiments, the concentration of resveratrol ester is from 1% to 5% or between 4% and 30%.

In some embodiments, the disclosed compositions may be administered in an oral dosage by tablet, capsule, gelcap, liquid or other common method of administration, optionally including another resveratrol ester analog or combination of resveratrol ester analogs in concentrations of from about 10 mg to about 1600 mg, such as 10 mg to 50 mg, 40 mg to 500 mg, or 400 mg to about 1600 mg.

In some embodiments, the disclosed compositions may be administered by injection by subcutaneous, intra muscular or intravenous route to maintain systemic resveratrol ester levels as a single analog or a combination of resveratrol ester analogs in a concentration of from about 5 mg to about 1600 mg, such as 5 mg to 100 mg, 90 mg to 200 mg, or 180 mg to about 1600 mg.

In some embodiments, the disclosed compositions may be administered in food products such as an energy bar, cereal, beverage, energy drink, etc., optionally containing one or more resveratrol ester analogs in concentrations of from about 0.1% to about 10%, such as at least 0.1% to 5% or 4% to about 10%.

Synthesis of 4AR

Air and moisture sensitive reagents were introduced via dry syringe or cannula. Toluene, xylene, pyridine, ethyl acetate, and N-methyl morpholine were distilled from CaH₂. DMF was dried by storage over 4 Å molecular sieves. Reagents were purchased from Aldrich and Lancaster. Flash chromatography was carried out using 60-230 mesh silica gel. Silica gel chromatography was performed using 1, 2, and 4 mm plates loaded with 230-400 mesh PF-254 gypsum bound silica. Analytical thin-layer chromatography (TLC) was performed with Merck silica gel 60 F₂₅₄, 0.25 mm pre-coated TLC plates. TLC plates were visualized using UV₂₅₄. All ¹H NMR spectra were obtained with 300 Varian spectrometers using TMS (0.00 ppm), chloroform (7.26 ppm), or acetone-d₆ (2.05 ppm) as an internal reference. Signals are reported as m (multiplet), s (singlet), d (doublet), t (triplet), q (quartet), and bs (broad singlet). ¹³C NMR were obtained with 75 MHz Varian spectrometer using TMS (0.0 ppm), Chloroform (77.2 ppm), or acetone-d₆ (30.6 ppm) as the internal standard. Mass spectra date (HRMS, EI) were obtained from the Brigham Young University mass spectrometry facility. Combustion analysis was performed by M-H-W Laboratories, Phoenix, Ariz.

4′-Acetoxy-resveratrol can be prepared according to Scheme 1.

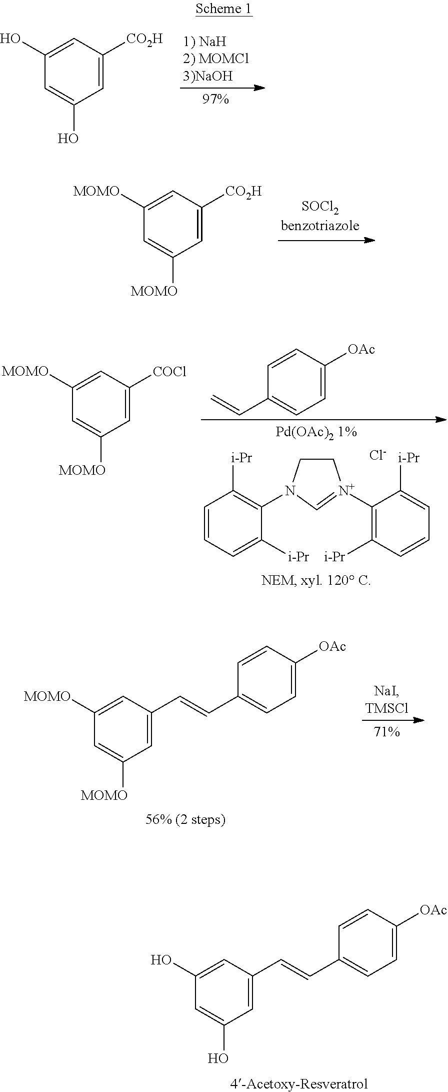

### 1. Preparation of 3,5-bis(methoxymethoxy)benzoic acid

A flame dried flask was charged with dry DMF (75 mL) and 60% oil dispersion NaH (3.8 g, 95 mmol). A solution of 3,5-dihydroxybenzoic acid (4.6 g, 30 mmol) in DMF (25 mL) was added dropwisely over 20 minutes. The mixture was allowed to stir for one hour under N₂. MOMCl (7.5 mL, 100 mmol) was added slowly so that the inner temperature did not exceed 50° C. After 30 hours, the insoluble material was filtered off and the filtrate was concentrated to an oil residue, which was partitioned between benzene and water. The water layer was extracted with benzene for another three times. The combined benzene extracts was dried over Na₂SO₄ and concentrated to pale yellow oil, which was dissolved in 50 mL methanol. 2N Aqueous NaOH (25 mL, 50 mmol) was added and the mixture was stirred for three hours. The mixture was concentrated and dissolved in 30 mL water. The aqueous solution was washed with benzene and acidified with 10% aqueous HCl. The precipitated white solid was filtered and washed with water and dried to give 6.6 g (91%) product, which was further purified by recrystallization from EtOAc-hexane. Data are: ¹H NMR (CDCl₃, 300 MHz) δ 7.44 (d, 2H), 6.98 (t, 1H), 5.21 (s, 4H), 3.50 (s, 6H); ¹³C NMR (CDCl₃, 75 MHz) δ 170.9, 158.4, 131.4, 111.5, 110.7, 94.7, 56.4; mp=129-130° C.; HRMS (EI⁺) found 242.0796 M⁺. calcd 242.0790 for C₁₁H₁₄O₆.

### 2. Preparation of 3,5-bis(methoxymethoxy)benzoyl chloride

A stock solution was prepared by dissolving benzotriazole (1.49 g, 0.0125 mmol), thionyl chloride (0.91 mL, 0.0125 mmol) in 8.0 mL DCM. Reaction was carried out by adding the stock solution intermittently to a stirred solution of 3,5-dimethoxybenzoic acid (2.42 g, 10 mmol) in 200 ml DCM. Before addition was complete, benzotriazole hydrochloride started precipitating out as a white solid. The mixture was stirred for another ten minutes. After filtration, the filtrate was stirred with MgSO₄7H₂O (5 g) to destroy excess thionyl chloride. The white solid was filtered off and the filtrate was concentrated to give 2.5 g (97%) crude product, which was used for the next step without further purification. Data are: ¹H NMR (CDCl₃, 300 MHz) δ 7.44 (d, 2H), 7.04 (t, 1H), 5.20 (s, 4H), 3.49 (s, 6H); ¹³C NMR (CDCl₃, 75 MHz) δ 168.1, 158.5, 135.3, 112.6, 111.9, 94.7, 56.4; HRMS (EI⁺) found 260.0465 M⁺. calcd 260.0452 for C₁₁H₁₃O₅Cl.

### 3. Preparation of 4′-acetoxy-3,5-bis(methoxymethoxy)stilbene

A 50 mL round bottom flask was charged with p-xylene (20 mL), Pd II catalyst (22.5 mg, 0.1 mmol), 1,3-bis-(2,6-diisopropylphenyl) imidazolinium chloride (42.7 mg, 0.1 mmol), 3,5-bis(methoxymethoxy)benzoyl chloride (2.42 g, 10 mmol), 4-acetoxystyrene (1.94 g, 12 mmol), and N-methyl morphorline (1.38 g, 12 mmol). The mixture was stirred at 120° C. for 3.5 h under nitrogen atmosphere. Then it was cooled to room temperature and EtOAc was added and filtered. The filtrate was washed with brine and dried over Na₂SO₄. Then it was filtered and purified via flash chromatography and gave the product (2.1 g, 59%) as a white solid. Data are: ¹H NMR (CDCl₃, 300 MHz) δ 7.48 (d, 2H), 7.08-6.93 (m, 4H), 6.86 (d, 2H), 6.66 (t, 1H), 5.19 (s, 4H), 3.50 (s, 6H), 2.30 (s, 3H); ¹³C NMR (CDCl₃, 75 MHz) δ 169.5, 158.7, 150.3, 139.5, 135.0, 128.7, 128.5, 127.6, 121.9, 108.0, 104.5, 94.6, 56.2, 21.2; HRMS (EI⁺) found 358.1409 M⁺. calcd 358.1416 for C₂₀H₂₂O₆

### 4. Preparation of 4′-acetoxy-3,5-dihydroxystilbene

To a solution of 4′-acetoxy-3,5-bis(methoxymethoxy)stilbene (0.358 g, 1 mmol) in dry DCM (50 mL) and dry CH₃CN (50 mL) were added NaI (1.8 g, 24 mmol) and freshly distilled TMSCl (1.52 g, 24 mmol). The mixture was stirred under argon for 15 minutes. The solution was diluted with DCM (50 mL) and washed with a fresh aqueous saturated solution of Na₂S₂O₃(3×40 mL) and saturated NaHCO₃, and water. The organic layer was dried over Na₂SO₄, filtered, and concentrated. The crude product was purified by flash column and gave 0.20 g product (72%) of 4′-acetoxy-resveratrol. Data are: ¹H NMR (Aceton-d₆, 300 MHz) δ 8.25 (s, 1H), 7.60 (m, 2H), 7.13-7.08 (m, 4H), 6.59 (d, 2H), 6.32 (t, 1H), 2.25 (s, 3H); ¹³C NMR (Aceton-d₆, 75 MHz) δ 169.7, 159.7, 151.4, 140.4, 136.0, 130.0, 128.3, 128.3, 123.0, 106.1, 103.3, 21.1; HRMS (EI⁺) found 270.0889 M⁺. calcd 270.0892 for C₁₆H₁₆O₄.

### Example 1

### Synthesis of 4AR

4′-Acetoxy-Resveratrol (referred to herein as 4AR) is synthesized in five steps beginning with resorcyclic acid, as described above (see PCT/US05/02229). Treatment with sodium hydride followed by methoxylmethyl chloride and exposure to sodium hydroxide gave approximately a 71% isolated yield. Purification of this molecule was then performed using standard chemical protocols.

The 4AR synthesized as described above demonstrated increased bioavailability, as shown by an increase in CLog P value from 2.833 for resveratrol to 3.687 for 4AR, which indicates increased stability and bioavailability at significantly. The stability and methods of synthesis result in a reduced cost to provide the desired compositions.

The synthetic method described above uses a direct, efficient synthesis at a low cost, and results in improved stability (longer shelf life) where it remains a white crystalline material for over 2 years, as well as higher biological activity (increase absorption, lower dose and longer half-life) with various cosmetic, nutraceuticals (nutritional supplements) and pharmaceutical applications, including (a) anti-aging, anti-oxidant, (b) anti-inflammatory, (c) anti-cancer, (d) neuroprotection (for improved brain health), (e) cardiovascular health, (f) prostate health and (g) skin health, as described below in the following examples.

### Example 2

### Effect of 4AR on Collagen Deposition

4′-Acetoxy-resveratrol was tested to determine whether it can stimulate collagen deposition in human monolayer dermal fibroblasts in the following examples. In this assay, we first determined the toxicity of the test materials (untreated cells, positive control-ascorbate, vehicle=0.1% DMSO, and a range of the 4′-acetoxy-resveratrol molecule, from approximately 10 nM to 10 μM).

Cytotoxicity was determined by spectrophotometric detection of reduced 3-(4,5-dimethlythiazol-2-yl)-2,5-diphenyltetrazolium bromide (MTT, Sigma Cat. #5655, lot #66H50336) at 550 nm. Metabolic activity can be used as a measure of cytotoxicity, in that the intensity of the reduced form of MTT by live cells is directly proportional to cell viability, and inversely proportional to cytotoxicity.

The human dermal monolayer fibroblasts were exposed to the test materials for approximately 48 hours. Thereafter, the samples were processed for MTT and collagen deposition, etc.

All test materials displayed similar MTT reduction (cytotoxicity) for the type of sample and dose range assayed (from approximately 10 nM to 10 μM). The number of replicates was 6 for each group.

Collagen deposition by ELISA for Human Type I collagen C-terminal propeptide was determined. Dermal fibroblasts synthesize primarily type I collagen, and the cleavage of the C-terminal propeptide is required for deposition into fibrils within the extracellular matrix. This propeptide can be measured using antisera which does not recognize the unprocessed from in cell culture supernatants, and is also used clinically as a measure of fibrosis in patient sera. The amount of cleaved propeptide is directly proportional to the amount of type I collagen deposited, and can be precisely quantified using purified standards and a commercial ELISA kit (Takara Mirus, Inc., Cat. # TAK-MK-101). After 48 hours in the presence of the test materials or controls, culture medium supernatants were removed and immediately analyzed using the ELISA kit according to manufacturer's instructions using a Molecular Devices Vmax plate 96 well plate reader and SoftMax software. Ascorbic acid (positive control; ascorbate, Sigma Cat. # A4544, lot #073K0139) was added to a final concentration of 20 μg/ml.

4AR at approximately 9 μM significantly increased collagen deposition in human dermal monolayer fibroblasts by 65% compared to vehicle control values.

These basic dermal data results show that 4AR is biologically active in skin parameters with the ability to significantly increase collagen deposition in human monolayer fibroblasts and may positively influence many other dermal components such as elastin, elastase, matrix metalloproteinases, collagenases, glycoaminoglycans and hyaluronic acid at the epidermal/dermal junction (see gene arrays studies, below). Additionally, the resveratrol analogs can bind the abundant distribution of beta estrogen receptors in the keratinocytes of the epidermis and fibroblasts in the dermis that also have a positive influence on skin parameters and enhanced dermal health.

### Example 3

### Effect of 4AR on Body Weight Gain

Adult male Long-Evans rats were administered 4AR for 21 days. DMSO served as the control and 4AR was dissolved into DMSO at the following concentrations: 5.0 mg/kg body weight, 20.0 mg/kg body weight and 90.0 mg/kg body weight (n=12 per group). There were no significant differences in food/water intake during the course of this study among the treatment groups.

After 10 days of receiving the treatments there was a significant decrease in body weight gain, by an average of approximately 17 grams, in the 90.0 mg/kg vs. the control values (525.0+5.1 vs. 542.1+6.2 grams, respectively. After 20 days on the treatments there was a significant decrease in body weight gain, by approximately 14 grams, in the 20.0 mg/kg vs. control values (566.0+4.7 vs. 551.8+3.7 grams). Finally, there was an even greater significant decrease in body weight gain between the 90.0 mg/kg vs. control values after 20 days on the treatments representing a 54.2 grams decrease (or 566.0+4.7 vs. 511.8+4.1 grams). These data suggest a positive influence of the resveratrol ester analog on body weight gain or weight control.

### Example 4

### Effect of 4AR on 5α-Reductase Enzyme Activity

Since, the 5a-reductase enzyme (which converts testosterone to 5a-dihydrotestosterone) is important in prostate health in reference to benign prostatic hyperplasia (BPH) and the treatment and prevention of prostate cancer, we determined if prostate type I 5α-reductase enzyme activity was impacted by these treatments by quantifying the enzymatic rates of each animal as described above (in this study there were approximately 10 animals per treatment group).

Table 1 shows the effect of 4AR on 5a-reductase enzyme activity as measured in pmol/hour incubation/mg of protein.

TABLE 1

| SAMPLE TYPE | ACTIVITY |
|---|---|
| DMSO-Controls | 8.7 + 0.6 |
| 5 mg/kg Body Weight (4AR) | 4.6 + 0.4* |
| 20 mg/kg Body Weight (4AR) | 3.3 + 0.3* |
| 90 mg/kg Body Weight (4AR) | 2.0 + 0.2* |

*denotes a significant decrease in 5α-reductase enzyme activity in the 4AR treated rats compared to the DMSO-control values.

These data show a significant and positive influence of the 4AR resveratrol analog on male prostate health as a potential treatment for benign prostatic hyperplasia (BPH) and the prevention of prostate cancer by significantly decreasing 5α-reductase enzyme activity.

### Example 5

### Gene Expression Studies

In this gene array experiment, we examined the ability of 4AR at a one percent concentration in DMSO (where 100% DMSO served as the vehicle) to either stimulate or inhibit several important genes and quantify directly the transcribed messenger RNA (mRNA) levels by Taqman real time PCR. The test material was placed onto a biological barrier (human dermal equivalent) using a MatTek “ready-to-use” in vitro organotypic tissue equivalent, based on well-documented and validated protocols, using the EpiDerm™ Model EPI-200 Kit. The MatTek kits uses normal (non-transformed) donated human cells as the basis for all of its tissue equivalents. These cells are grown (cultured) in standard Millipore Millicell™ Single Well Tissue Culture Plate Inserts at the air liquid interface or ALI. Apical (top) surface of tissue is exposed to air allowing for direct application of test articles. DMSO only treated samples served as controls along with untreated samples. Once the 4 AR test material penetrated the human skin barrier equivalent the gene chip was activated and subsequently mRNA levels were quantified and the results represent stimulation or inhibition of a specific gene.

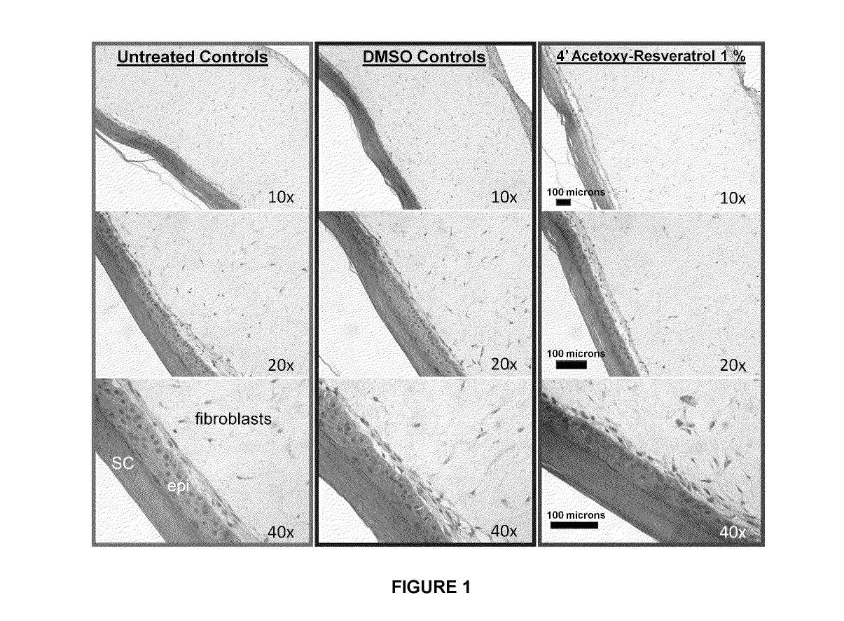

The in vitro human dermal tissue model that served as the biological barrier in these gene array studies is shown in FIG. 1.

To investigate the effects of 4AR on the expression of genes associated with the extracellular matrix (ECM) of the skin, qPCR experiments were conducted using the tissue cultures (model system described above) with subsequent quantification of mRNA levels from neurosensory tissue/cells. There are many genes involved in skin health that include ECM proteins and several other gene products that are involved in inflammation, anti-oxidant activity, anti-aging, regulators of ECM factors and DNA repair (Baumann LS, 2009, Cosmetic Dermatology: Principles and Practice, Revised 2nd edition, McGraw-Hill, Columbus, Ohio, USA.; Wolff K, Goldsmith L, Katz S et al (eds), 2008, Collagen, Elastic Fibers, and Extracellular Matrix of the Dermis. In: Fitzpatrick's Dermatology in General Medicine 7th edition, McGraw-Hill, Columbus, Ohio, USA, Chapter 61). A dose of 1.0% of 4AR dissolved in 100% DMSO along with the other control treatments (outlined above) were exposed to the human equivalent skin model for 24 hours.

The results of a gene array study examining 4AR as an example of a novel topical agent for cosmetic applications displayed below in Summary Table 2.

TABLE 2
Gene Function Summary

| | | | Assay result |
|---|---|---|---|
| Gene Marker | Reported Role | Reporting Reference(s) | with 4AR |
| A. Anti-Aging | Anti-aging, biological | http://genomics.senescence.info/ | SIRT1 |
| Marker: SIRT1 | activity on human skin | genes/entry.php?hgnc=SIRT1 | activity |
| (Sirtuin 1) | cells activates the | http://findarticles.com/p/articles/mi_m0PDG/is_6_6/ | increased by |
| | nuclear longevity protein | ai_n19328744/pg_4/?tag=content;col1 | 3.3-fold |
| | SIRT1, improving DNA | | |
| | integrity and acts as a | | |
| | anti-senescence marker | | |
| B. Collagen/ECM | Play significant roles in | http://www3.interscience.wiley.com/ | Tissue |
| formation and | wound repair | journal/90510948/abstract | inhibitor of |
| breakdown: | Elevates collagen levels | http://www.jbc.org/content/270/7/3423.abstract | metalloproteinases 1 |
| Tissue inhibitor of | Youth marker | | increased by |
| metalloproteinases 1 | | | 2.5-fold |
| B. Collagen/ECM | Major component in | http://www.pnas.org/content/85/24/9679.abstract | Type IV |
| formation and | basement membranes | | collagen |
| breakdown: Type | Involved in collagen | | alpha 1 |
| IV collagen alpha 1 | production | | increased by |
| | | | 1.7-fold |
| B. Collagen/ECM | Major component in | http://www.pnas.org/content/85/24/9679.abstract | IV collagen |
| formation and | basement membranes | | alpha 2 |
| breakdown: Type | Involved in collagen | | increased by |
| IV collagen alpha 2 | production | | 1.8-fold |
| B. Collagen/ECM | Promotes collagen that | http://askville.amazon.com/Cheap-place-buy-Epicuren- | Procollagen |
| formation and | is found in youthful skin | Pro-Collagen-III/AnswerViewer.do?requestId=11938437 | III increased |
| breakdown: | Anti-wrinkle | | by 2.2-fold |
| Procollagen III | | | |
| (alpha 1) | | | |
| B. Collagen/ECM | Youth marker, increased | Jo Seltzer and Arthur | TIMP2 |
| formation and | by Vitamin C | Eisen, 2003, The Role of | increased by |
| breakdown: | | Extracellular Matrix | 1.3-fold |
| Tissue Inhibitor of | | Metalloproteinases in | |
| metalloproteinases | | Connective Tissue | |
| 2 (TIMP2) | | Remolding, In: | |
| | | Fitzpatrick's Dermatology | |
| | | in General Medicine 6th | |
| | | edition, McGraw-Hill, New | |
| | | York, NY, USA, Chapter | |
| | | 17 | |
| B. Collagen/ECM | Regulates MMP 1 | http://en.wikipedia.org/wiki/MMP1 | Protein |
| formation and | expression which breaks | | Kinase C |
| breakdown: | down interstitial | | alpha |
| Protein Kinase C | collagens | | increased by |
| alpha | | | 2.7-fold |
| B. Collagen/ECM | A protein in connective | http://en.wikipedia.org/wiki/Elastin | Elastin |
| formation and | tissue that is elastic and | | increased by |
| breakdown: Elastin | allows many tissues in | | 2.8-fold |
| | the body to resume their | | |
| | shape after stretching or | | |
| | contracting. Elastin | | |
| | helps skin to return to its | | |
| | original position when it | | |
| | is poked or pinched | | |
| C. Inflammation: | Proinflammatory | http://en.wikipedia.org/wiki/Interleukin | IL-6 |
| IL-6 (interleukin 6) | | | decreased by |
| | | | 35.2-fold |
| C. Inflammation: | Proinflammatory | http://www.genecards.org/cgi- | IL-1a |
| IL-1a (interleukin | marker of irritancy | bin/carddisp.pl?gene=II1a | decreased by |
| 1a) | Induces apoptosis | | 10.1-fold |
| C. Inflammation: | Proinflammatory | http://en.wikipedia.org/wiki/Interleukin_8 | IL-8 |
| IL-8 (interleukin 8) | especially in response to | | decreased by |
| | psoriasis | | 3.8-fold |
| C. Inflammation: | Proinflammatory, | http://www3.interscience.wiley.com/journal/ | S100 |
| S100 Calcium | especially in psoriasis | 118568631/abstract?CRETRY=1&SRETRY=0 | Calcium |
| binding protein A8 | | | binding |
| | | | protein A8 |
| | | | decreased by |
| | | | 2.7-fold |
| C. Inflammation: | Proinflammatory | http://books.google.com/books?id=5XKs5ACErHgC&pg= | IL-1RII |
| IL-1RII (interleukin | | PA352&lpg=PA352&dq=il-1rii+in+skin&source= | decreased by |
| 1RII) | | bl&ots=k9KOhgWNdm&sig= | 1.9-fold |
| | | AwTEv80VjTiajNa4Zk1BR8WDxbM&hl= | |
| | | en&ei=QWbJSsvAGlu_lAfUjZmSAw&sa= | |
| | | X&oi=book_result&ct=result&resnum= | |
| | | 2#v=onepage&q=il-1rii%/20in%20skin&f=false | |
| C. Inflammation: | Proinflammatory | 14. cyclooxygenase-2: an | COX 2 |
| COX 2 (PTGS2) | | enzyme that makes | decreased by |
| | | prostaglandins that cause | 1.7-fold |
| | | inflammation and pain and | |
| | | fever; | |
| | | wordnetweb.princeton.edu/perl/webwn | |
| C. Inflammation: | Proinflammatory, | http://www3.interscience.wiley.com/ | S100 |
| S100 Calcium | especially in psoriasis | journal/118568631/abstract?CRETRY=1&SRETRY=0 | Calcium |
| binding protein A9 | | | binding |
| | | | protein A9 |
| | | | decreased by |
| | | | 1.6-fold |
| C. Inflammation: | Induces apoptosis | http://www.genecards.org/cgi-bin/ | TNFRSF1A |
| TNFRSF1A | Proinflammatory | carddisp.pl?gene=TNFRSF1A | decreased by |
| (Tumor necrosis | Increases with age | | 1.4-fold |
| factor receptor | | | |
| superfamily, | | | |
| member 1A) | | | |
| D. Anti-oxidant: | redox and oxidative | http://genomics.senescence.info/ | Superoxide |
| Superoxide | regulation | genes/entry.php?hgnc=SOD1 | dismutase 1, |
| dismutase 1, | Antioxidant activity | | soluble |
| soluble | | | increased by |
| | | | 1.6-fold |
| D. Anti-oxidant: | redox and oxidative | http://genomics.senescence.info/ | Superoxide |
| Superoxide | regulation | genes/entry.php?hgnc=SOD2 | dismutase 2, |
| dismutase 2, | Anti-oxidant activity | | mitochondrial |
| mitochondrial | | | increased by |
| | | | 1.7-fold |
| D. Anti-oxidant: | cross-linking collagen | http://en.wikipedia.org/wiki/Lysyl_oxidase | Lysyl oxidase |
| lysyl oxidase | and elastin | | increased by |
| (LOX) | | | 1.9-fold |
| D. Anti-oxidant: | Antioxidant agent | http://www.accessmylibrary.com/coms2/ | Metallothionein 2 |
| Metallothionein 2 | | summary_0286-31734665_ITM | increased by |
| | | | 3.4-fold |
| D. Anti-oxidant: | Antioxidant agent | http://www.accessmylibrary.com/coms2/ | Metallothionein 1 |
| Metallothionein 1 | | summary_0286-31734665_ITM | increased by |
| | | | 64.0-fold |
| D. Anti-oxidant: | Antioxidant, prevents | http://www.rcsb.org/pdb/static.do?p=education_discussion/ | Catalase |
| Catalase | free radical damage | molecule_of_the_month/pdb57_1.html | increased by |
| | | | 1.6-fold |
| E. Growth factors: | tumor suppressor gene | http://en.wikipedia.org/wiki/ | TGF beta |
| TGF (transforming | This receptor/ligand | TGF_beta_receptor_2 | type II |
| growth factor) beta | complex phosphorylates | | receptor |
| type II receptor | proteins, which then | | increased by |
| | enter the nucleus and | | 1.7-fold |
| | regulate the transcription | | |
| | of a subset of genes | | |
| | related to cell | | |
| | proliferation | | |
| | Induces procollagen I | | |
| | and III | | |
| | Reduced after UV | | |
| | exposure | | |
| E. Growth factors: | Growth factor, especially | http://stke.sciencemag.org/cgi/ | HBEGF |
| HBEGF (Heparin- | in healing wounds in | content/abstract/develop;132/19/4317 | increased by |
| binding EGF-like | epithelial tissue | | 1.9-fold |
| growth factor) | | | |
| E. Growth factors: | controls proliferation, | http://en.wikipedia.org/wiki/TGF_beta | TGF beta |
| TGF (transforming | cellular differentiation | | increased by |
| growth factor) beta | acts as an | | 2.0-fold |
| | antiproliferative factor in | | |
| | normal epithelial cells | | |
| | and at early stages of | | |
| | oncogenesis | | |
| | induces procollagen I | | |
| | and III | | |
| E. Growth factors: | stimulates growth | http://genomics.senescence.info/ | IGF1 |
| IGF1 (Insulin-like | | genes/entry.php?hgnc=IGF1 | increased by |
| growth factor 1) | | | 2.6-fold |
| E. Growth factors: | A neurotrophin important | http://genomics.senescence.info/ | Nerve growth |
| Nerve growth | for the development and | genes/entry.php?hgnc=NGF | factor |
| factor | maintenance of the | A Micera et al. PNAS, | increased by |
| | central nervous system, | 2001, 98: 6162-6167 | 6.7-fold |
| | NGF stimulates division | | |
| | and differentiation | | |
| | Growth and | | |
| | development | | |
| | PNS development | | |
| | Involved in tissue repair | | |
| F. Cell adhesion/ | Desmocollin 1 is part of | http://www.in-cosmeticsasia.com/ExhibitorLibrary/ | Desmocollin |
| desmosomes: | a desmosomal cell | 59/Skin_rejuvenation_with_a_biomimetic_pep- | 1 decreased |
| Desmocollin 1 | adhesion receptor | tide_designed_to_promote_{—-} | by 2.4-fold |
| | formed in terminally | desquamation_SOFW_2006_3.pdf | |
| | differentiating | | |
| | keratinocytes of stratified | | |
| | epithelia. | | |
| | Overexpression in old | | |
| | skin (a decrease would | | |
| | be a sign of youth) | | |
| F. Cell adhesion/ | Desmosomes are cell- | http://www.in-cosmeticsasia.com/ExhibitorLibrary/ | Desmoglein |
| desmosomes: | cell junctions between | 59/Skin_rejuvenation_with_a_biomi- | 3 decreased |
| Desmoglein 3 | epithelial, myocardial, | metic_peptide_designed_to_promote_desquama- | by 1.9-fold |
| | and certain other cell | tion_SOFW_2006_3.pdf | |
| | types. Desmoglein 3 is a | http://en.wikipedia.org/wiki/ | |
| | calcium-binding | Desmoglein_3 | |
| | transmembrane | | |
| | glycoprotein component | | |
| | of desmosomes in | | |
| | vertebrate epithelial | | |
| | cells. | | |
| G. NF-KB/AP-1 | Decreased in aged skin | http://www.sciencedirect.com/science?_ob= | JunD |
| Signaling | | ArticleURL&_udi=B6T6J-4JFHF2T- | increased by |
| Pathways: JunD | | 2&_user=456938&_rdoc= | 2.1-fold |
| | | 1&_fmt=&_orig= | |
| | | search&_sort=d&_docanchor= | |
| | | &view=c&_searchStrId= | |
| | | 1059942232&_rerunOrigin= | |
| | | google&_acct=C000021830&_version= | |
| | | 1&_urlVersion=0&_userid= | |
| | | 456938&md5=ae5efdcd07b60060a081832dbc5f2f0c | |
| G. NF-KB/AP-1 | The FOS oncogene | http://genomics.senescence.info/ | Fos |
| Signaling | plays an important role | genes/entry.php?hgnc=FOS | increased by |
| Pathways: Fos | in development, cell | | 3.4-fold |
| (FBJ murine | proliferation, and | | |
| osteosarcoma viral | differentiation | | |
| oncogene) | Decreased in aging skin | | |
| G. NF-KB/AP-1 | regulators of cell | http://en.wikipedia.org/ | FosB |
| Signaling | proliferation, | wiki/FOSB | increased by |
| Pathways: FosB | differentiation, and | | 8.2-fold |
| (FBJ murine | transformation | | |
| osteosarcoma viral | Decreased in aging skin | | |
| oncogene | | | |
| homolog B) | | | |
| H. Cellular Growth/ | A photomarker for aging | http://www3.interscience.wiley.com/ | Fibrillin |
| regeneration: | skin so helps skin stay | journal/118683054/abstract | increased by |
| Fibrillin | younger and healthier | | 1.3-fold with |
| | | | 4AR |
| H. Cellular Growth/ | Glycoprotein important | http://www.antibodies-online.com/ | Laminin 1B |
| regeneration: | for basement membrane | antigen/laminin,+gamma+1+ | increased by |
| Laminin 1B | health | (formerly+LAMB2)+(LAMC1)/ | 1.4-fold with |
| | | | 4AR |
| H. Cellular Growth/ | Repair of DNA, | http://www.pubmedcentral.nih.gov/ | PCNA |
| regeneration: | decreased in aged skin | articlerenderfcgi?artid=1483034 | increased by |
| PCNA | | http://escience.invitrogen.com/ipath/ | 5.4-fold with |
| (Proliferating Cell | | iPath.jsp?cn= | 4AR |
| Nuclear Antigen) | | United%20States&mapid= | |
| | | 427&highlightGene=PCNA | |
| H. Cellular Growth/ | Increased in aged skin | http://www.molecularstation.com/research/ | CK 2E |
| regeneration: | | protein-kinase-ck2-is-a-key-activator- | decreased by |
| Cytokeratin 2E | | of-histone-deacetylase-in-hypoxia- | 3.5-fold with |
| | | associated-tumors-17935135.html | 4AR |

1-9. (canceled)

10. A method of providing enhanced resveratrol activity comprising administering a therapeutically effective amount of 4-acetoxy-resveratrol.

11. The method of claim 10, wherein 4-acetoxy-resveratrol is administered in a topical cream, lotion, gel or other cosmetic formulation, wherein the topical cream, lotion, gel or other cosmetic formulation optionally further comprises another resveratrol ester analog or combination of resveratrol ester analogs in a concentration between 0.3% and 12%.

12. The method of claim 10, wherein 4-acetoxy-resveratrol is administered in a dermal patch delivery system, wherein the dermal patch delivery system optionally further comprises another resveratrol ester analog or combination of resveratrol ester analogs in a concentration between 1% and 30%.

13. The method of claim 10, wherein 4-acetoxy-resveratrol is administered in an oral dosage, wherein the an oral dosage optionally further comprises another resveratrol ester analog or combination of resveratrol ester analogs in an amount between 10 mg and 1600 mg.

14. The method of claim 10, wherein 4-acetoxy-resveratrol is administered in by injection, wherein the injection optionally further comprises another resveratrol ester analog or combination of resveratrol ester analogs in an amount between 5 mg and 1600 mg.

15. The method of claim 10, wherein 4-acetoxy-resveratrol is administered in a food product, wherein the food product optionally further comprises another resveratrol ester analog or combination of resveratrol ester analogs in a concentration between 0.1% and 10%.

16. A method of treating or preventing conditions mediated by sirtuins, comprising administering a therapeutically effective amount of 4-acetoxy-resveratrol.

17. The method of claim 16, wherein 4-acetoxy-resveratrol is administered in a topical cream, lotion, gel or other cosmetic formulation, wherein the topical cream, lotion, gel or other cosmetic formulation optionally further comprises another resveratrol ester analog or combination of resveratrol ester analogs in a concentration between 0.3% and 12%.

18. The method of claim 16, wherein 4-acetoxy-resveratrol is administered in a dermal patch delivery system, wherein the dermal patch delivery system optionally further comprises another resveratrol ester analog or combination of resveratrol ester analogs in a concentration between 1% and 30%.

19. The method of claim 16, wherein 4-acetoxy-resveratrol is administered in an oral dosage, wherein the an oral dosage optionally further comprises another resveratrol ester analog or combination of resveratrol ester analogs in an amount between 10 mg and 1600 mg.

20. The method of claim 16, wherein 4-acetoxy-resveratrol is administered in by injection, wherein the injection optionally further comprises another resveratrol ester analog or combination of resveratrol ester analogs in an amount between 5 mg and 1600 mg.

21. The method of claim 16, wherein 4-acetoxy-resveratrol is administered in a food product, wherein the food product optionally further comprises another resveratrol ester analog or combination of resveratrol ester analogs in a concentration between 0.1% and 10%.

22. A method of treating or preventing conditions mediated by estrogen and anti-estrogen hormone actions comprising administering a therapeutically effective amount of 4-acetoxy-resveratrol.

23. The method of claim 22, wherein 4-acetoxy-resveratrol is administered in a topical cream, lotion, gel or other cosmetic formulation, wherein the topical cream, lotion, gel or other cosmetic formulation optionally further comprises another resveratrol ester analog or combination of resveratrol ester analogs in a concentration between 0.3% and 12%.

24. The method of claim 22, wherein 4-acetoxy-resveratrol is administered in a dermal patch delivery system, wherein the dermal patch delivery system optionally further comprises another resveratrol ester analog or combination of resveratrol ester analogs in a concentration between 1% and 30%.

25. The method of claim 22, wherein 4-acetoxy-resveratrol is administered in an oral dosage, wherein the an oral dosage optionally further comprises another resveratrol ester analog or combination of resveratrol ester analogs in an amount between 10 mg and 1600 mg.

26. The method of claim 22, wherein 4-acetoxy-resveratrol is administered in by injection, wherein the injection optionally further comprises another resveratrol ester analog or combination of resveratrol ester analogs in an amount between 5 mg and 1600 mg.

27. The method of claim 22, wherein 4-acetoxy-resveratrol is administered in a food product, wherein the food product optionally further comprises another resveratrol ester analog or combination of resveratrol ester analogs in a concentration between 0.1% and 10%.

28. A method of treating or preventing a skin condition comprising administering a therapeutically effective amount of 4-acetoxy-resveratrol.

29. The method of claim 28, wherein 4-acetoxy-resveratrol is administered in a topical cream, lotion, gel or other cosmetic formulation, wherein the topical cream, lotion, gel or other cosmetic formulation optionally further comprises another resveratrol ester analog or combination of resveratrol ester analogs in a concentration between 0.3% and 12%.

30. The method of claim 28, wherein 4-acetoxy-resveratrol is administered in a dermal patch delivery system, wherein the dermal patch delivery system optionally further comprises another resveratrol ester analog or combination of resveratrol ester analogs in a concentration between 1% and 30%.

31. The method of claim 28, wherein 4-acetoxy-resveratrol is administered in an oral dosage, wherein the an oral dosage optionally further comprises another resveratrol ester analog or combination of resveratrol ester analogs in an amount between 10 mg and 1600 mg.

32. The method of claim 28, wherein 4-acetoxy-resveratrol is administered in by injection, wherein the injection optionally further comprises another resveratrol ester analog or combination of resveratrol ester analogs in an amount between 5 mg and 1600 mg.

33. The method of claim 28, wherein 4-acetoxy-resveratrol is administered in a food product, wherein the food product optionally further comprises another resveratrol ester analog or combination of resveratrol ester analogs in a concentration between 0.1% and 10%.
